(19) United States
(12) Patent Application Publication
(10) Pub. No.: US 2024/0172964 A1
(43) Pub. Date: May 30, 2024

(54) System and Method for Processing Ergonomic Data

(71) Applicant: Bayerische Motoren Werke Aktiengesellschaft, Muenchen (DE)

(72) Inventors: Arman DEHGHANI, Muenchen (DE); Patrick Frenzel, Borsdorf (DE); Fabian Guenzkofer, Kirchdorf a.d. Amper (DE); Kristina Schreyer, Muenchen (DE); Marc Snell, Muenchen (DE)

(21) Appl. No.: 18/517,074

(22) Filed: Nov. 22, 2023

(30) Foreign Application Priority Data
Nov. 25, 2022 (DE) 10 2022 131 294.1

(51) Int. Cl. A61B 5/11; A61B 5/22

(52) U.S. Cl. CPC A61B 5/1116; A61B 5/1114; A61B 5/1118; A61B 5/22

(57) ABSTRACT

A system for processing ergonomic data includes a reception unit configured to receive first ergonomic data from a first detection device and second ergonomic data from a second detection device. The first and second ergonomic data are mutually independent. The system also includes a processing unit. The processing unit is configured to assign the first ergonomic data to the second ergonomic data in an automated manner and, on the basis of mutually assigned data, to execute a further processing of ergonomic data thus assigned.

### CROSS REFERENCE TO RELATED APPLICATION

This application claims priority under 35 U.S.C. § 119 from German Patent Application No. 10 2022 131 294.1, filed Nov. 25, 2022, the entire disclosure of which is herein expressly incorporated by reference.

### BACKGROUND AND SUMMARY

The present invention relates to a system for processing ergonomic data comprising a reception unit, which is configured to receive first ergonomic data from a first detection device and second ergonomic data from a second detection device, wherein the first ergonomic data and the second ergonomic data are mutually independent. The present invention further relates to a method for processing ergonomic data comprising capturing first ergonomic data and second ergonomic data, wherein the first and second ergonomic data are mutually independent.

In various technical fields, particularly in production, it is necessary for loads acting on persons working in the locality to be minimized. A key aspect is the ergonomics of workstations and movement sequences. At present, to a major extent, ergonomic evaluation of loads acting on the body is not automated. Analysis of physical postures is generally executed by reference to a subjective evaluation of joint angles, and of the duration and frequency of body positions. If technical facilities (e.g. a force gauge) are employed for the measurement of loads, the resulting raw data, for the further appraisal thereof, undergoes manual processing and a subjective evaluation. This evaluation involves a complex subjective appraisal of physical posture and directions of force. In particular, various information on physical posture and force measurement are processed and evaluated separately.

The object of the present invention is therefore to permit an automated and comprehensive processing of ergonomic data.

This object is fulfilled by a system for processing ergonomic data and by a method for processing ergonomic data according to the claimed invention.

The system for processing ergonomic data comprises a reception unit which is configured to receive first and second ergonomic data. In particular, the first and second ergonomic data originate from a first detection device for the detection of first ergonomic data, and from a second detection device for the detection of second ergonomic data. The two detection devices, and thus also the first and second ergonomic data, are preferably mutually independent. This means that, by way of entirely separate devices, two different data streams can be generated and received by the reception device. The first and second detection devices can be arbitrary devices, which are capable of detecting ergonomic data for a user. In particular, by way of the first and second detection devices, two different types of ergonomic data are detected, such as, for example, motion and force.

It should be observed that, although the first and second detection devices deliver separate ergonomic data, the two detection devices can be integrated in a single and superordinate primary device, which relays ergonomic data captured to the system proposed herein. The first and second ergonomic data can also be relayed to the system in a single data stream, which comprises both the first and the second ergonomic data. Moreover, the first and second ergonomic data can also be supplied by a single detection device. Moreover, it is also possible for the system to receive and process three or more ergonomic data sets and/or that three or more detection devices are provided.

The system further comprises a processing unit, which is configured to assign the first ergonomic data to the second ergonomic data in an automated manner and, on the basis of mutually assigned data, to execute a further processing of ergonomic data thus assigned. Thus, in comparison with an existing system, rather than the separate processing and evaluation of different ergonomic data, ergonomic data from two different detection devices are combined, and are only evaluated thereafter. This combination, and preferably also evaluation, proceeds in an automated manner, i.e. with no subjective consideration by a person. In this manner, a more rapid and more objective evaluation of ergonomic data is permitted.

The processing unit can preferably execute the combination of the first and second ergonomic data, and the subsequent evaluation thereof, by the employment of a machine learning algorithm. In particular, the processing unit itself can be implemented in the form of a machine learning algorithm, in particular as a neural network.

According to one embodiment, the first detection device comprises one or more sensors, which are configured to capture a physical posture and/or a motion of a user. The first detection device can be, for example, a motion capturing system, which can represent the position of a body, i.e. the body or parts of the body of a user, in a three-dimensional space. This can be executed, either by way of sensors which are fitted to the body, or by way of one or more cameras, or by a combination of both. For example, a first detection device of this type can comprise one or more inertial measurement units (IMUs). In turn, these measurement units contain various sensors such as, for example, accelerometers, gyroscopes and magnetometers. These measurement units, particularly in a wireless arrangement, can be fastened to a body of a user by way of clothing, e.g. T-shirts, or by fastening devices, e.g. Velcro strips. Further to the positioning and calibration of measurement units, the first detection unit captures the position, orientation and/or acceleration of various or individual body segments. This information can then be translated into a digital human model.

First ergonomic data then indicate the physical posture and/or motion of the user. Both physical posture and motion can be captured by the above-mentioned measurement units and, in particular, are represented by a combination of information from measurement units. First ergonomic data can either comprise multiple data streams containing individually captured information, such as position, orientation and/or acceleration, or can directly comprise the digital human model.

According to a further embodiment, the second detection device is configured to execute a force measurement. To this end, the second detection device comprises one or more sensors, in order to detect a force which is exerted by the user. For example, the second detection device can be a force-sensing glove or similar, which is capable of measuring forces exerted in the hand and finger region, e.g. during assembly operations.

Second ergonomic data can thus indicate a force exerted, in particular a magnitude, direction and/or duration of the force exerted. In particular, multiple force values can be indicated, which are captured at various monitored positions of a body part of the user.

First and second ergonomic data thus captured by the detection devices can be transmitted to the reception unit in a wireless or hard-wired arrangement. For example, the detection devices and the reception unit can mutually communicate via a wireless connection, in order to transmit first and second ergonomic data.

For the assignment of first and second ergonomic data, the processing unit, according to one embodiment, can be configured to execute a temporal assignment of data. In this manner, information from first ergonomic data captured at a specific time point can be assigned to information from the captured second ergonomic data. This means, for example, that a force is assigned to a captured motion and/or physical posture which is respectively exerted during said motion and/or physical posture. Thus, during an assembly process, not only the force immediately exerted or the physical posture immediately assumed by a user are detected. In existing systems, it has been detected, or subjectively evaluated whether a physical posture assumed fulfils ergonomic conditions, or whether the force exerted falls within the scope of ergonomic conditions. To date, however, a combined evaluation as to whether, in a specific posture, both ergonomic conditions for posture and for the force exerted are fulfilled has not been possible in an automated manner. Now, however, on the basis of first and second ergonomic data, the processing unit can execute an automated assignment of various information with respect to physical posture or motion, and with respect to force exerted. Correspondingly, a subsequent automated evaluation of data is also possible.

If the first and second detection devices deliver two separate data streams, which are not temporally synchronized, the processing unit can be configured to firstly synchronize the first and second ergonomic data. This synchronization can be omitted, if the two detection devices, or at least the data delivered by the latter, are already synchronized.

In order to synchronize first and second ergonomic data, the processing unit can be configured to detect a predefined motion in the first and second ergonomic data which are captured by the first and second detection devices. A motion of this type can be executed by the user beforehand, i.e. prior to the actual capture of data. This is preferably a motion which does not form part of the subsequently executed movement sequence, such that it can be unambiguously detected. This motion can thus include both a specific and unambiguously identifiable physical posture, and an unambiguously identifiable exertion of force. For example, a motion of this type can be a handclap, preferably above the head. Other unambiguously identifiable motions are also possible.

According to a further embodiment, the processing unit is configured to execute the mutual temporal synchronization of first and second ergonomic data on the basis of the time point of the detection of the predefined motion. For example, the two data streams of first and second ergonomic data can be mutually temporally offset, such that the time point of the detection of the predefined motion occurs at the same time. A time axis of a data stream can also be displaced, such that both data streams, at the time point of the predefined detected motion, indicate the same time. The respective time in both data streams for the time point of the detected predefined motion can also be set to zero, and time counted from this point forward. In each case, both data streams can then be processed in combination wherein, in the first and second ergonomic data, motions and forces captured at the same time points also occurred simultaneously in practice.

As described above, the two detection devices are preferably separate devices and can comprise, for example, measurement systems from different manufacturers. In such a case, the detection devices themselves employ different software for the processing of data and, customarily, there is no direct communication between detection devices. However, time-synchronized information from both measurement systems represent a precondition for a comprehensive ergonomic analysis. In particular, whole-body forces and the handling loads require an evaluation of both physical posture and of a force/pulse value. For these criteria, it is necessary for a user to know e.g. which posture was assumed for the exertion of a specific force, or in which direction a force was exerted. However, the first detection device for the capture of physical posture and/or motion, in isolation, will not be aware when a force is exerted, whereas the second detection device for the capture of force will have no information on direction or posture. By the synchronization of data and/or the assignment of data, however, the system described herein can superimpose force data upon physical posture and/or orientation of motion, thereby rectifying omissions which are inherent to each detection device.

According to a further embodiment, the processing unit is configured, on the basis of processed ergonomic data, to execute an ergonomic evaluation. Such an evaluation can be executed on the basis of known information, which indicates which motion at which force fulfils an ergonomic criterion, i.e. whether or not the force exerted and/or the associated motion exceed or undershoot permissible threshold values. In particular, the combination of force and motion can be evaluated, i.e. whether or not the force exerted, for an assigned motion/physical posture, exceeds or undershoots a saved and predefined threshold value.

According to a further aspect, a method is proposed for processing ergonomic data, wherein the method comprises the following steps: capture of first ergonomic data and of second ergonomic data, wherein the first and second ergonomic data are mutually independent, assignment of the first ergonomic data to the second ergonomic data and, on the basis of mutually assigned data, a further processing of ergonomic data.

As described above with reference to the system, by way of the method described herein, an automated processing and evaluation of ergonomic data are permitted, which data originate from different and separate detection devices. To date, data from such detection devices have been evaluated subjectively, as data streams between existing digital detection systems have not been synchronized, such that no overall ergonomic detection has been possible. However, this results in a less reliable and more complex ergonomic evaluation. This is improved by the method described wherein, in this case, ergonomic data from two separate detection devices are firstly mutually assigned and, if necessary, synchronized, and a common evaluation of ergonomic data is executed thereafter. This evaluation which, in particular, includes a comparison with saved threshold values, can thus involve various types of ergonomic data, in the present case, in particular, both a force and an associated physical posture or motion, i.e. the direction of force.

As described above with reference to the processing unit, the method can also be implemented in the form of a machine learning algorithm, and particularly as a neural network. Various algorithms can be employed for this purpose, which are capable of executing the method described.

Embodiments and features of the proposed system for processing ergonomic data apply correspondingly to the proposed method.

A computer program product is further proposed, comprising program code which is configured to initiate the execution on a computer of the above-mentioned method.

A computer program product can be provided or supplied, for example, in the form of a storage medium, such as e.g. a memory card, a USB stick, CD-ROM or DVD, or in the form of a downloadable data file from a network server. This can be executed, e.g. in a wireless communication network, by the transmission of a corresponding data file containing the computer program product.

Further potential implementations of the invention also include combinations, which are not specifically indicated, of above-mentioned features or embodiments, or of those described hereinafter with reference to the exemplary embodiments. A person skilled in the art will also add individual aspects by way of improvements or additions to the respective basic form of the invention.

Further advantages and advantageous embodiments are disclosed in the description, the drawings and the claims. In particular, combinations of features disclosed in the description and in the drawings are exemplary only, such that features can also be provided in isolation, or in different combinations.

The invention is described in greater detail hereinafter, with reference to the exemplary embodiments represented in the drawings. Exemplary embodiments, and combinations represented in the exemplary embodiments, are exemplary only, and do not define the protective scope of the invention.

### BRIEF DESCRIPTION OF THE DRAWINGS

FIG. 1 shows a schematic view of a system for processing ergonomic data.

FIG. 2 shows an exemplary diagram of data for a motion capture.

FIG. 3 shows an exemplary diagram of data for a force measurement.

### DETAILED DESCRIPTION OF THE DRAWINGS

Hereinafter, identical or functionally equivalent elements are represented by the same reference symbols.

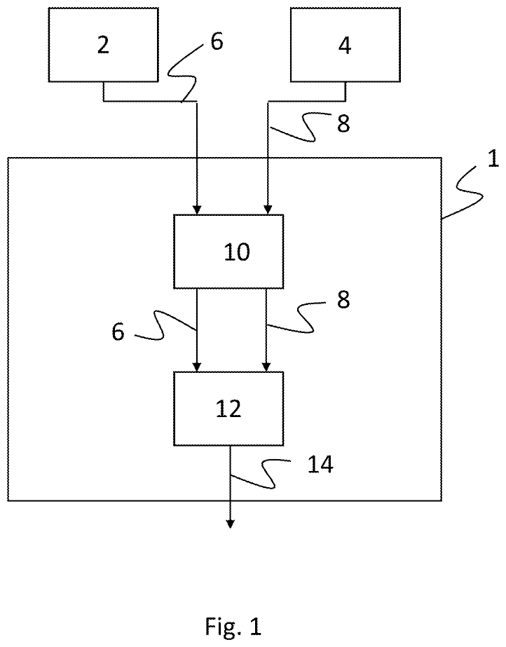

FIG. 1 shows a system 1 for processing ergonomic data. Such a system 1 can be employed, for example, in production, in order to check and evaluate the ergonomics of existing workstations. For the capture of various ergonomic data, such as motion, force, physical posture, etc., detection devices 2, 4 can be employed. As represented in FIG. 1, these detection devices 2, 4 can be provided separately from the system 1, and can communicate with a reception unit 10 of the system 1. Alternatively, the detection devices 2, 4 can also be integrated directly in the system. Moreover, the detection devices 2, 4 can also be embodied as a single device, which captures the first and second ergonomic data 6, 8 and relays the latter, for example as a single data stream, to the reception unit 10.

To date, such detection devices 2, 4 have been employed only for the determination of individual aspects of ergonomics, e.g. motion capture for physical postures and force-sensing gloves for the determination of assembly forces. Such applications capture instantaneous records of a specific posture or force, but require a user who understands the context of the working task concerned.

The system 1 described herein now provides an automated processing of ergonomic data 6, 8 from two detection devices 2, 4, and an automated evaluation of these data 6, 8. Alternatively, the evaluation of data 6, 8 can be executed in a further subsequent system and process.

For the processing and evaluation of ergonomic data 6, 8, ergonomic data are firstly captured by the detection devices 2, 4. The two detection devices 2, 4 are separate devices which, in particular, deliver two or more different types of ergonomic data 6, 8. The first detection device 2 can capture, for example, a physical posture and/or motion, and deliver the latter as first ergonomic data 6. This can be executed by way of sensors, which are fastened to a user, or by way of cameras or similar. The second detection device 4, in turn, can capture, for example, a force exerted, and deliver the latter as second ergonomic data 8. A force measurement or capture of this type can also be executed by way of sensors which are fastened to a user. For example, the second detection device 6 can be a force-sensing glove.

First and second ergonomic data 6, 8 are received in the system 1 by the reception unit 10. To this end, the reception unit 10 can communicate with the detection units 2, 4, for example via a wireless connection. This provides an advantage, in that the detection units 2, 4 can be located on the site of detection, e.g. on the production line or on the user, and the reception unit 10 can be arranged remotely, in particular with further elements of the system 1.

In the interests of the improved processing and evaluation of ergonomic data 6, 8, a processing unit 12 is provided. In particular, this delivers an automated processing and evaluation of ergonomic data 6, 8.

Automated processing firstly comprises an automated assignment of first ergonomic data 6 to second ergonomic data 8. This means that, e.g. a captured force is assigned to a captured physical posture. Accordingly, a physical posture assumed and the force exerted can no longer be determined in a separate manner only. Instead, a force is assigned to a specific motion, or a direction is assigned to a force. It is thus possible to determine, for example, the force which is applied by a user for the execution of a specific action, such as the snap-fitting of a component. This combined information 14 can then be employed by the processing unit 12 in order to determine whether this specific action, such as the snap-fitting of a component, is executed with an ergonomically acceptable physical posture and force, or whether an improvement is required. Alternatively, the processing unit 12 can relay combined data 14, optionally with an evaluation, to further systems or units.

Conversely to existing systems, in the system 1 described herein, firstly, the capture and processing of ergonomic data 6, 8 are thus executed in an automated manner and, secondly, ergonomic data 6, 8 are not processed and evaluated separately, but are firstly combined or mutually assigned, and only evaluated thereafter in the form of overall information.

In order to permit the mutual assignment of data 6, 8, it can be necessary for the two detection devices 2, 4 or data streams 6, 8 to be mutually temporally synchronized. Further to such synchronization, for a specific exerted force, information with respect to which is included in the second data 8, at a specific time point, the associated physical posture or motion can be read-out in a simple manner from the first data 6 at the same synchronized time point.

In order to synchronize the two data streams 6, 8, a user can execute a predefined motion, which can be identified in both data streams 6, 8. For example, this can be a handclap above the head. In particular, the predefined motion is a motion which does not customarily form part of the movement sequence which is otherwise detected.

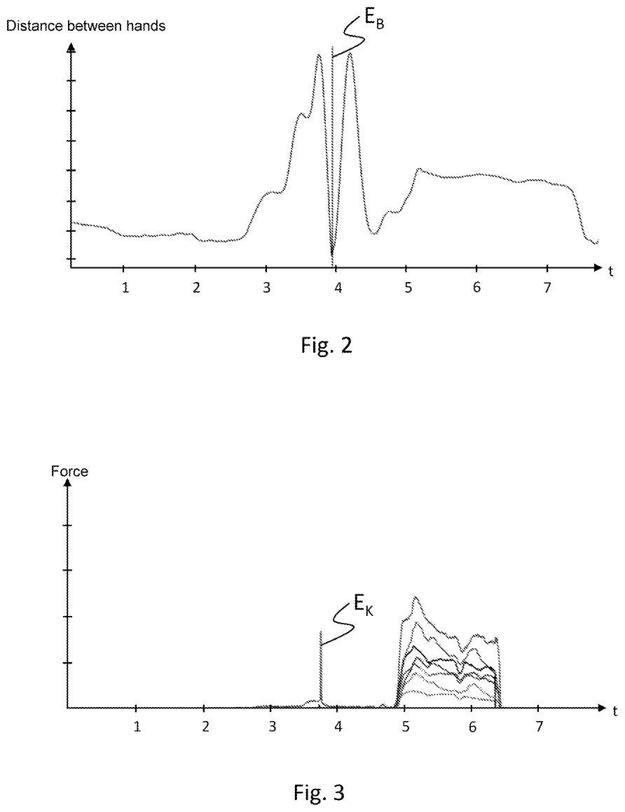

FIGS. 2 and 3 show exemplary diagrams of captured data, wherein FIG. 2 represents a captured motion, in this particular case a distance between the hands, and FIG. 3 represents a measured force, plotted against time in each case.

In FIG. 2, for the detection of the predefined clapping motion, a distance between the hands is monitored. The handclap is thus characterized by a minimum point between two maxima. The minimum point is then considered as the synchronization event E_{B}, and the associated time is employed for the purposes of synchronization.

In FIG. 3, in a simple manner, the synchronization event E_{K} can be detected as the first peak, i.e. as the force associated with the predefined motion, given that, at this time point, a clearly visible force is exerted for the first time.

The predefined motion, which is employed as the synchronization event E_{B}, E_{K}, respectively occurs at different times in the respective data streams 6, 8. If the two data streams 6, 8 are not synchronized, a reliable assignment of motion to force is therefore not possible.

The processing unit 12 is therefore configured to detect the predefined motion or synchronization event E_{B}, E_{K} in the captured ergonomic data or data streams 6, 8. Further to the detection of motion E_{B}, E_{K}, the processing unit 12 can then execute the respective matching or mutual synchronization of both data streams 6, 8, or the time stamps thereof. In future, in both data streams 6, 8, information for a specific time point can thus be read-out and evaluated in the form of respectively assigned or mutually associated information.

By way of the system described herein, it is thus possible, in a simple manner, for different ergonomic data, in particular from different ergonomic detection devices, to be processed and mutually combined in an automated manner. To this end, inter alia, machine learning algorithms can be employed. The system thus permits an ergonomic evaluation which is based upon a plurality of different ergonomic data.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

### REFERENCE SYMBOLS

1 System
2 First detection device
4 Second detection device
6 First ergonomic data
8 Second ergonomic data
10 Reception unit
12 Processing unit
14 Superimposed/combined data
E_{B} Synchronization event for motion
E_{K} Synchronization event for force

What is claimed is:

1. A system for processing ergonomic data, the system comprising:
a reception unit, which is configured to receive first ergonomic data from a first detection device and second ergonomic data from a second detection device, wherein the first ergonomic data and the second ergonomic data are mutually independent, and
a processing unit, wherein the processing unit is configured to assign the first ergonomic data to the second ergonomic data in an automated manner and, based on mutually assigned data, to execute a further processing of the assigned data.

2. The system according to claim 1, wherein the first ergonomic data indicate a physical posture and/or a motion of a user.

3. The system according to claim 1, wherein the second ergonomic data indicate a force which is exerted by the user.

4. The system according to claim 3, wherein the second ergonomic data indicate a magnitude, direction and/or duration of the force.

5. The system according to claim 1, wherein the processing unit is further configured to execute a mutual assignment of the first ergonomic data and the second ergonomic data which are captured at a specific time point.

6. The system according to claim 5, wherein a force exerted is assigned to a captured motion and/or physical posture.

7. The system according to claim 1, wherein the processing unit is further configured to synchronize the first ergonomic data and the second ergonomic data.

8. The system according to claim 1, wherein the processing unit is further configured to detect a predefined motion in the first ergonomic data and the second ergonomic data.

9. The system according to claim 8, wherein the processing unit is further configured to execute mutual temporal synchronization of the first ergonomic data and the second ergonomic data based on a time point of the predefined motion.

10. The system according to claim 1, wherein the processing unit is further configured to execute an ergonomic evaluation based on the assigned data.

11. The system according to claim 10, wherein the processing unit is further configured to execute the ergonomic evaluation based on saved threshold values.

12. A method for processing ergonomic data, the method comprising:
capturing first ergonomic data and second ergonomic data, wherein the first and second ergonomic data are mutually independent, and
assigning the first ergonomic data to the second ergonomic data and, based on mutually assigned data, further processing of the assigned data.
